(19) United States
(12) Patent Application Publication
(10) Pub. No.: US 2007/0185533 A1
(43) Pub. Date: Aug. 9, 2007

(54) Neurofeedback conditioning method

(75) Inventor: Lee Gerdes, Scottsdale, AZ (US)

Correspondence Address: WEISS & MOY PC, 4204 NORTH BROWN AVENUE, SCOTTSDALE, AZ, 85251, US

(73) Assignee: BRAINSTATE TECHNOLOGIES, INC.

(21) Appl. No.: 11/347,169

(22) Filed: Feb. 3, 2006

(51) Int. Cl. A61N 1/39

(52) U.S. Cl. 607/1

(57) ABSTRACT

An improved method for conditioning the brain for optimal cognitive functioning based on the use of neurofeedback. Using an EEG, a subject's brain activity is continually analyzed. A neurofeedback program is established for the subject on an automated individualized basis, based on the immediate brain activity and the understanding of optimized cognitive functioning. The subject is then exposed to sound waves at frequencies sufficient to alter brain activity from the immediate brain state when that brain state is not optimized, to a brain state that is more conducive to being optimized for cognitive functioning.

### FIELD OF THE INVENTION

This invention relates generally to neurofeedback conditioning methods and, more particularly, to an improved neurofeedback conditioning method that seeks to automatically balance brain waves in an individualized manner, consistent with optimized cognitive performance. Cognitive functioning refers to the mental processes by which knowledge is acquired. These mental processes include perception, reasoning, acts of creativity, problem-solving and possible intuition.

### BACKGROUND OF THE INVENTION

The high amount of stress in daily life is caused by increased demands on our time. Persons in the work force are being asked to produce more in a shorter amount of time, family activities are heavily scheduled, and the amount of electronic stimuli to which persons in developed countries are exposed has grown exponentially in the last several decades. The intensity to accomplish more in less time is the basic component for stress in daily life. Stress causes the brain to tighten; oxygen is utilized from the blood more slowly, and brain functioning and processing slows. In general, stress causes reduced efficiency of the brain power required to accomplish cognitive functioning.

There are a great number of activities known to help reduce stress—breathing exercises, regular physical exercise, meditation, yoga, and others. However, these activities are for the purpose of reducing stress after it has already manifested, rather than preventing stress from having such a damaging affect on optimized brain functioning. Stress affects a person as if the individual who has stress is a container and stress is a substance that is poured into it. The stress-reducing activities help “pour out” the stress from the container, or person. These activities are helpful as responses to an over-full container. They are not helpful to reduce the container from getting full in the first place. These activities empty the container rather than prevent it from being filled—they are prescriptive rather than preventative. And, after the person is affected by stress to a significant degree, the person then experiences decreased cognitive functioning, which often then causes even greater stress, with the result that the container gets more stress poured into it. This merry-go-round effect leads the stress spiral to poorer and less efficient cognitive performance. Additionally, these prior art stress reducing methods do not condition the brain so that the brain is aware that the stress response to certain stimuli is unhelpful to it's optimized functioning.

Brain activity creates electromagnetic energy—captured and observed as brain waves with EEG amplifiers and computers—which indicate how the brain is functioning. Brain activity is based on neurons which interact and connect with each other to form groups known as “neuro-nets”. These neuro-nets are activated based on stimuli. As a consequence, when a certain stimulus is experienced—like a mouse jumping out from behind a counter—we have a brief moment of fear and jump back. This occurs because neuro-nets were activated that created a pathway for us to jump, for our hearts to race a bit, and possibly for us to utter a noise in response to such stimulus.

Neurofeedback, which exposes a person to sound waves at certain predetermined frequencies, has also been used to deal with brain functioning. For example, one prior art neurofeedback method is based on a Quantitative Electroencephalographic Analysis (QEEG). Using a QEEG, the neurofeedback provider compares the brain waves of the client to a normative data base of other brain waves. Following such comparison, irregularities are noted for neurofeedback training. A significant problem with a QEEG is the basic assumption that the database of brain waves is helpful to establish a normal or healthy brain wave pattern for an individual.

A need exists for an improved neurofeedback method that is individualized, in order to more effectively condition the brain while it is working to be optimized for cognitive functioning. The present invention satisfies this need and provides other, related, advantages.

### SUMMARY OF THE INVENTION

In accordance with an embodiment of the present invention, a brain conditioning method is disclosed for an automated optimized cognitive functioning. The method comprises automatically determining a subject's baseline brain activity as reflected in electromagnetic waves emitted by the subject's brain, establishing an individualized feedback program for the subject based on exposure of the subject to sound waves from an external source, exposing the subject to sound waves automatically generated from the external source at frequencies sufficient to alter brain activity when the dominant brain frequency does not fall in a range expected for optimal cognitive functioning, and repeating the exposing step until the brain activity is altered to be more optimized for cognitive functioning.

### BRIEF DESCRIPTION OF THE DRAWINGS

FIG. 1 is a flow chart, illustrating steps in a neurofeedback method consistent with an embodiment of the present invention.

### DETAILED DESCRIPTION OF THE PREFERRED EMBODIMENTS

Initially, it should be noted that the method of the present invention requires the use of certain basic hardware and software. In one embodiment, the method utilizes EEG electrodes; an EEG amplifier; a computer device; and software loaded on to the computer and configured to operate as disclosed herein.

An artist may want to be able to tap into realms of increased creativity. Corporate managers may want to become more positive and creative leaders. Sales professionals may want to become more attentive and respond more appropriately to the customer's position. Students, particularly those preparing for major examinations, may wish to optimize their cognitive functioning to enhance their capability to study.

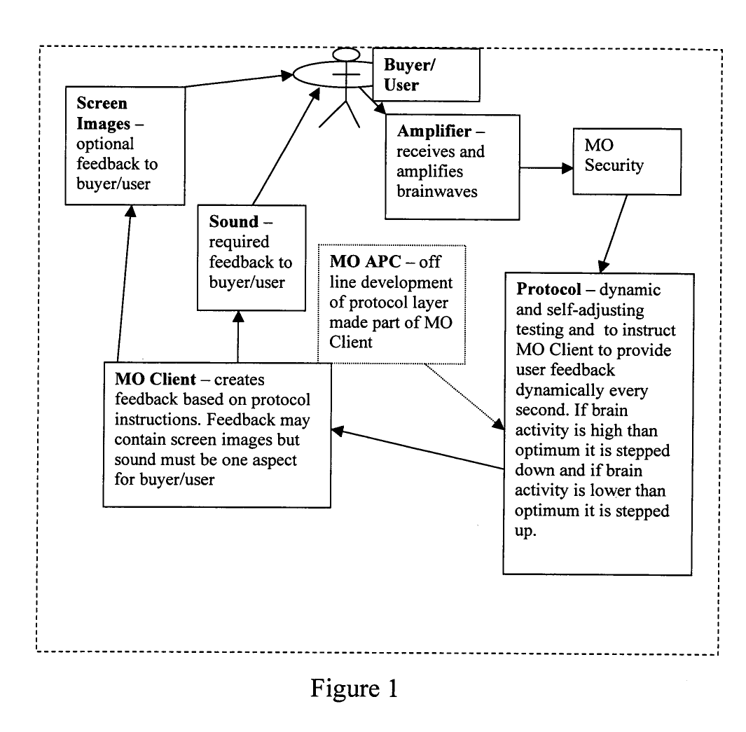

Referring now to FIG. 1, in one embodiment, current brainwave activity is assessed automatically and dynamically and then feedback is presented back to the subject.

More particularly, feedback is provided to the brain in the form of sound waves. These sound waves are higher frequency than the brain waves when the brain is functioning in a given second with a dominant frequency which is lower than is optimized for a cognitive functioning brain; and the sound wave feedback is produced at a lower frequency than the dominant brain wave frequency when that dominant frequency is found to be higher than is optimized for a cognitive functioning brain. Overall, the goal is generally the creation of a more balanced brain state.

The positive feedback in the form of sound waves is provided to the subject via headphones. During each elapsed second of training, the note that is selected is higher than the dominant brain frequency when the dominant brain frequency is lower than optimal; and lower than the dominant brain frequency when the dominant brain frequency is higher than optimal. The note is preferably between 5 Hz and 11 Hz different than the dominant brain frequency.

In this regard, if some brain waves are encouraged and others are discouraged, the brain will begin to move toward a more balanced condition. This balance will happen based only on the brain wave changes—and these changes can be encouraged by watching the brain waves and listening to the wave based sound feedback. This balancing—or conditioning—of the brain waves, balances them into optimized patterns.

An analogy to what is being accomplished with the method of the present invention can be found with tuning forks and the principle of resonance. Resonance accounts for the fact that when two tuning forks of the same frequency are placed in close proximity to each other, both will produce a sound even if only one of them is struck. Like a tuning fork, when the brain hears sounds when it generates or inhibits certain frequencies, other parts of the brain respond—resonate—to the frequency of the sound that is heard. Soon, the brain balances itself to the desired frequency, and thereby cuts new neuro-net pathways.

It is preferred to repeat the brain state conditioning as herein described, so that the desired effects on brain state can be more permanently achieved. Usually, subjects recognize benefits in the first one to three sessions. A brain state conditioning training session usually lasts a maximum of 20 minutes. Sessions may be completed twice every day. Usually, subjects train 6 minutes twice a day on the first day, 8 minutes twice a day the second day, and add 2 minutes to each training session a day until they reach 20 minutes per session. Subjects will usually train for two weeks straight, and then train 3 days per week for an additional 4 to 10 weeks depending on the subject.

### Statement of Operation

The first step in the brain conditioning method of the present invention is for electrodes to be connected to a subject at T3 and T4 (the left and right temple areas of the scalp as defined by the EEG 10-20 International System). It should be clear that although in the preferred embodiment the subject is human, substantial benefit could be derived from an alternative configuration of the present invention in which the subject is non-human, such as another primate. The electromagnetic waves emitted by the subject's brain are monitored by a computer. The subject's baseline brain activity is determined by the computer through a software program. An individualized feedback program for the subject is dynamically established by the software based on exposing the subject to a combination of sound waves from an external source. Once calculated by the computer program, the subject is exposed to sound waves automatically generated from the external source and delivered to the subject through headphones. The software instructs the external source to emit a first set of dynamic sound waves at higher frequencies than the subject's dominant brain activity when the subject's dominant brain activity is lower than 9 Hz and at lower frequencies than the subject's dominant brain activity when the subject's dominant brain activity is higher than 16 Hz. The software also instructs the external source to emit a second set of dynamic sound waves, preferably musical notes, at frequencies sufficient to reduce a total amplitude of all of the subject's brainwaves outside of a range of approximately 9 Hz to 16 Hz. The first set of sound waves is continuously exposed to the subject in order to entrain the subject's brain activity into the range of approximately 9 Hz to 16 Hz. The second set of sound waves are intermittently exposed to the subject in order to reduce the total amplitude of the all of the subject's brainwaves outside of a range of approximately 9 Hz to 16 Hz. In this way, the first set of sound waves and the second set of sound waves combine to adjust a subject's brain state into a brain state optimized for cognitive functioning.

While the invention has been particularly shown and described with reference to preferred embodiments thereof, it will be understood by those skilled in the art that the foregoing and other changes in form and details may be made therein without departing from the spirit and scope of the invention.

I claim:

1. A brain conditioning method to optimize cognitive functioning comprising:
automatically determining a subject's baseline brain activity as reflected in electromagnetic waves emitted by the subject's brain;
establishing an individualized feedback program for the subject based on exposure of the subject to sound waves from an external source;
exposing the subject to sound waves automatically generated from the external source at frequencies sufficient to alter brain activity when the dominant brain frequency does not fall in a range expected for optimal cognitive functioning; and
repeating the exposing step until the brain activity is altered to be more optimized for cognitive functioning.

2. The method of claim 1 wherein the step of determining the subject's current brain activity is accomplished by placing electrodes at T3 and T4 and monitoring activity detected by the electrodes.

3. The method of claim 1 wherein the external feedback source is a pair of headphones.

4. The method of claim 1 wherein the exposing step comprises providing sound waves when one of low frequencies are dominant and high frequencies are dominant.

5. The method of claim 1 wherein the exposing step further comprises playing musical notes when a combination of high and low frequency amplitudes is in a declining state.

6. The method of claim 5 wherein the exposing step further comprises playing a higher musical note when low frequency dominates, and a lower musical note when high frequency dominates.

7. The method of claim 1 wherein automatically determining the subject's baseline brain activity comprises monitoring a range of frequencies between approximately 3 Hz and 40 Hz.

8. The method of claim 7 further comprising the step of determining a dominant brain frequency within said range of frequencies by identifying which frequency has the greatest amplitude.

9. The method of claim 1 wherein a range expected for optimal cognitive functioning being between approximately 9 Hz and 16 Hz.

10. The method of claim 1 wherein the exposing step is repeated until the dominant brain activity falls within a range of approximately 9 Hz and 16 Hz and the amplitude of low frequencies and high frequencies is reducing.

11. The method of claim 1 wherein the subject is exposed to a first set of sound waves automatically generated from said external source at frequencies sufficient to at least one of lower a subject's high dominant frequency and raise a subject's low dominant frequency while at the same time said subject is exposed to a second set of sound waves automatically generated from said external source at frequencies sufficient to reduce a total amplitude of all said subject's brainwaves outside of a range of approximately 9 Hz to 16 Hz.

12. The method of claim 11 wherein said second set of sound waves being musical notes.
